(19) United States
(12) Patent Application Publication
(10) Pub. No.: US 2026/0165605 A1
(43) Pub. Date: Jun. 18, 2026

(54) METHOD AND SYSTEM FOR PROVIDING POSITION INFORMATION OF AN OBJECT

(71) Applicant: Siemens Healthineers AG, Forchheim (DE)

(72) Inventor: Hendrik Paysen, Erlangen (DE)

(21) Appl. No.: 19/421,057

(22) Filed: Dec. 16, 2025

(30) Foreign Application Priority Data
Dec. 18, 2024 (DE) 10 2024 212 094.4

(51) Int. Cl. A61B 5/11; A61B 5/055; G01R 33/30; G01R 33/44

(52) U.S. Cl. CPC A61B 5/1114; A61B 5/055; G01R 33/307; G01R 33/445

(57) ABSTRACT

A method for providing position information of an object in a patient tunnel of magnetic resonance tomograph includes: arranging the object outside the patient tunnel; moving the object inward into the patient tunnel along an axis of symmetry of a B0 field magnet of a magnetic resonance tomograph; acquiring a magnetic resonance signal for a predefined slice during inward movement of the object into the patient tunnel; and determining the position information of the object based on the acquired magnetic resonance signal.

The present patent document claims the benefit of German Patent Application No. 10 2024 212 094.4, filed Dec. 18, 2024, which is hereby incorporated by reference in its entirety.

### TECHNICAL FIELD

The present disclosure relates to a method and to a system for providing position information of an object in a patient tunnel of a magnetic resonance tomograph, to a system for providing such position information, and to a corresponding computer program element.

### BACKGROUND

Magnetic resonance tomographs (MRTs) are image-generating apparatuses that, for the purposes of imaging an object under examination, orient nuclear spins of the object under examination with a strong external magnetic field and excite them to precess around the orientation by an alternating magnetic field. The precession or return of the spins from the excited state into a lower-energy state in turn generates in response an alternating magnetic field that may be received via antennas. Spatial encoding may be impressed onto the signals with the assistance of magnetic gradient fields, the encoding subsequently permitting assignment of the received signal to a volume element. The received signal is then evaluated and a three-dimensional image depicting the object under examination is provided.

The radio-frequency fields used during a magnetic resonance examination may result in patient heating. In order to be able to provide patient safety, specific absorption rate (SAR) limit values have been defined, in particular for the head, the exposed body, and the entire body. Models with which the absorbed power may be assigned to the various body parts of a patient have been created for monitoring these limit values. For example, it is known to use a cylinder model, in which the patient's anatomy may be approximated with the assistance of cylinders for the head, torso, and legs and the absorbed power for the respective cylinder may be calculated or estimated for a uniform B field. However, to maximize the accuracy of modeling, it is necessary to be aware of positions of a patient's body regions, (e.g., of the head), in relation to the isocenter of the magnetic resonance tomograph. For example, it is known to determine a patient's head position by measuring the head position manually. Such a head position is in particular measured in relation to a patient table, the position of which is known and on which the patient is lying.

It has been found in this connection that there is a further need to provide a method and a system with which it is possible to provide position information of an object, in particular a patient's head position, in a patient tunnel of a magnetic resonance tomograph.

### SUMMARY AND DESCRIPTION

It is therefore an object of present disclosure to provide a method and a system with which it is possible to provide position information of an object, (e.g., a patient's head position), in a patient tunnel of a magnetic resonance tomograph.

The scope of the present disclosure is defined solely by the appended claims and is not affected to any degree by the statements within this summary. The present embodiments may obviate one or more of the drawbacks or limitations in the related art.

According to the disclosure, a method for providing position information of an object in a patient tunnel of a magnetic resonance tomograph includes: arranging the object outside the patient tunnel; moving the object inward into the patient tunnel along an axis of symmetry of a B0 field magnet of the magnetic resonance tomograph; acquiring a magnetic resonance signal for a predefined slice during inward movement of the object into the patient tunnel; and determining the position information of the object on the basis of the acquired magnetic resonance signal.

In other words, the present disclosure proposes using a magnetic resonance tomograph for signal acquisition in a predefined slice during inward movement of the object into the patient tunnel. In this way, it is possible to acquire the moment at which the object has reached the predefined slice during inward movement because it is only when the object has moved into the predefined slice that a strong magnetic resonance signal is detected. As soon as the object may be detected in the predefined slice, it is known that the object is located in/at the position of the predefined slice and therefore the position of the object with regard to the axis of symmetry of the B0 field magnet may be determined on this basis.

Such a procedure may be carried out when determining a patient's head position when the patient is moved inward into the patient tunnel in “head-first” position. Head-first may be taken to mean that it is the patient's head that is first moved inward into the patient tunnel. In such a case, a strong magnetic resonance signal is obtained as soon as the head reaches the predefined slice. If, on the other hand, the patient is moved inward into the patient tunnel in a “feet-first” arrangement, the position of the foot may be correspondingly established.

It is additionally also possible to establish the head position in a “feet-first” arrangement by detecting the moment or position at which a magnetic resonance signal may no longer be detected, in other words the moment at which the patient's body is moved out of the predefined slice. Since the method proposed here may provide as it were a profile of a patient during inward movement, it is also possible to determine positions or regions of other body parts. All that is required in this respect is to further evaluate the magnetic resonance signal and in particular to correlate/match it with the patient's body shape.

A patient's head position and/or foot position may be determined by the proposed procedure. These positions may then be used for modeling the specific absorption rates. The specific absorption rates may thus be modeled comparatively accurately irrespective of whether a patient has been correctly positioned on the patient table and also irrespective of possible measurement errors in the event of manual measurement of a patient's head or foot position. Furthermore, the proposed procedure may be comparatively simply integrated into workflows during a patient examination as the patient table on which the patient is positioned may be moved into the patient tunnel at the beginning of an examination. The proposed method may be carried out during this movement of the patient table such that the duration of the examination is not increased or not substantially so as a result.

The term “patient tunnel” may be interpreted broadly here. In particular, patient tunnel may refer to the interior of the “tube” of magnetic resonance tomographs. Alternatively, the term patient tunnel may also refer to the sensitive region suitable for imaging of a magnetic resonance tomograph of any desired configuration.

The term “object” may be interpreted broadly here. In particular, an object may be taken to mean a human or animal body or a part thereof. An object may be a patient's head, a patient's feet, and/or any desired determinable/differentiable body part of a patient, such as the neck, knees, or the like.

The phrase “position information of an object” may be interpreted broadly here and may be indicated by a point on the Z coordinate axis or also as a region on the Z coordinate axis. For example, the position information may be the beginning of a patient's head on the Z coordinate axis. It is alternatively or additionally possible to indicate a region in which a patient's head is located, with a Z coordinate for the beginning of the head and a Z coordinate at the end of the head. The end of a patient's head may take place, for example, by detecting a neck region, at the beginning of which the magnetic resonance signal may decline.

The phrase “arranging the object” may likewise be interpreted broadly here and may include positioning a patient on a patient table. Moving the object inward into the patient tunnel may take place by a continuous movement of the patient table into the patient tunnel, wherein non-continuous movements are also included here.

Spatial encoding is conventionally based on an X-Y-Z coordinate system. The Z coordinate axis is conventionally and, as here, defined as an axis of symmetry of a B0 field magnet of the magnetic resonance tomograph through a patient tunnel of the B0 field magnet in the preferential direction of the B0 field. In the conventional setup of a magnetic resonance tomograph, the Z coordinate axis is oriented horizontally and extends centrally through the opening of the windings of the B0 field magnet through a capture region of the B0 field magnet. The object to be captured is conventionally introduced into the patient tunnel parallel to the Z coordinate axis on a mobile patient table. Together with the Z coordinate axis, an X coordinate axis and a Y coordinate axis enclose a space, wherein the coordinate axes may be provided orthogonal to one another and the X coordinate axis is oriented horizontally and the Y coordinate axis vertically.

In certain examples, no switching of gradients in the predefined slice or slice plane, i.e., in the plane perpendicular to the slice selection direction, is performed during a sequence for acquiring the magnetic resonance signal for the predefined slice or during the performance of the disclosed method. “Gradient” may refer to a linear gradient field that may be switched in for spatial encoding. In particular, no read-out gradients and no phase-encoding gradients are switched. These gradients are hereinafter also denoted X or Y gradients because, in certain embodiments, they are at least roughly oriented along the X and Y coordinate axis (or vice versa). As a result, only a one-dimensional (1D) data set is captured in each case since no spatial encoding takes place within the slice. Since in principle only 1D spatial information is required in the Z coordinate axes, the X and Y gradients may be deactivated, i.e., the X and Y gradients need not be used for the present method. As a result, the acoustic noise concomitant with X and Y gradient switching may be avoided. Furthermore, fewer or no currents are induced in the object, such that the method does not give rise to any additional SAR exposure. Signal noise caused by X and Y gradient switching may also be avoided.

Z gradient switching of the magnetic resonance tomograph may be carried out at a constant amplitude for a predefined duration, wherein the amplitude may be located in a range of 1 to 4 mT/m, in a range of 1.5 to 3 mT/m, or in a range of 2.0 to 2.5 mT/m. The Z gradient may be set to a constant amplitude for the entire run time of the sequence. As a result, once a constant Z amplitude has been switched on, there is no need when carrying out the present method for further Z gradient switching such that no signal noise and also no corresponding acoustic noise is caused by a switch change.

The object may be moved inward into the patient tunnel at a speed in a range of 0.5 cm/s to 5 cm/s, at a speed in a range of 1.0 cm/s to 3.5 cm/s, or at a speed of 2.0 cm/s. The object may be bedded on a patient table that may be advanced into the patient tunnel. Moving the object inward into the patient tunnel at such a speed may in particular be provided by appropriately controlling an actuating mechanism of the patient table.

The predefined slice may be a transverse slice that is selected at least substantially orthogonally to the direction of movement of the object. “At least substantially” may be taken to mean that the orientation of the slice may deviate from the transverse or orthogonal by up to ±10° or up to ±5°. A transverse slice may be excited by loading a radio-frequency pulse while simultaneously switching a Z gradient. The slice selection gradient may also contain slight X and/or Y gradients.

The predefined slice may be centrally arranged, i.e., at Z=0, because the B0-magnetic field is particularly uniform and the slice therefore particularly flat in this position. However, the position may differ therefrom, for example, the slice may be located in a range of Z=−20 cm to Z=+20 cm. Positioning at the edge of the sensitive region makes it possible to detect when the object enters this region. Slice thickness may be in a range of 2 mm to 2 cm. Since the purpose of the sequence is merely to detect the presence of an object, slice thickness is of no concern. Slice thickness may be selected such that as little energy as possible may be used for radio-frequency excitation. Slice thickness may be selected in a range of 2 mm to 20 mm, in a range of 5 mm to 15 mm, or at 10 mm.

In certain examples, radio-frequency excitation is performed with just one radio-frequency pulse per repetition time (TR). In other words, just one single radio-frequency pulse may be used per repetition time. This selected radio-frequency pulse may be comparatively weak such that the radio-frequency power is negligible with regard to patient heating and therefore the specific absorption rates need not be monitored for carrying out the present method. The radio-frequency pulse may have a relatively small bandwidth. In certain examples, the phase of the radio-frequency pulse varies from TR to TR.

The sequence for acquiring the magnetic resonance signal may be very simple, for example, one radio-frequency pulse per repetition time (TR) followed by an acquisition window (TA).

The acquisition window (TA) may be located in a range of 2 ms to 15 ms, in a range of 3 ms to 10 ms, or at 4 ms in length. The repetition time (TR) may be located in a range of 500 ms to 2000 ms, in a range of 800 ms to 1500 ms, or at 1000 ms.

As already explained above, the position information may indicate the beginning or the end of the object along the axis of symmetry of the B0 field magnet of the magnetic resonance tomograph. The present disclosure is, however, not limited thereto. The object may be a patient's head or foot and the position information indicates the patient's head and/or foot position, e.g., at the beginning or the end of the head or a foot in the Z coordinate axis.

In certain examples, the present method is used for the determination of a patient's head position so as to be able to provide as accurate a (cylinder) model of a patient as possible such that the respective limit values for absorbed power may be correspondingly monitored thereby. The present disclosure is, however, not limited to this use case. Instead, the position data determined by the present disclosure may also be put to use in other use cases in which the position of a portion of patient anatomy is required. For example, consistency checks may also be carried out using the determined position data and appropriate data processing acts. It is, for example, possible to establish whether a patient's profile corresponds to a human anatomy.

The position information may be determined or converted in relation to the patient table such that the object's determined position information may straightforwardly be used for controlling a patient table. A further X-Y-Z coordinate system oriented relative to the patient table may be introduced for this purpose with the Z coordinate axis, for example, being oriented along the longitudinal axis of the patient table, the X coordinate axis may be horizontally and the Y coordinate axis may be vertically, wherein the coordinate axes are again provided orthogonal to one another. Such a coordinate system oriented relative to a patient table is denoted in practice a “table coordinate system” (TCS).

The disclosure furthermore relates to a system for providing position information of an object in a patient tunnel of a magnetic resonance tomograph. The system includes: a processing circuit; a (non-transitory) storage medium; and a data interface. The (non-transitory) storage medium includes a computer program with instructions that, on execution of the program, cause the processing circuit to carry out the above-described method, wherein the data interface is set up to receive the magnetic resonance signal for a central transverse slice of the object. The above explanations regarding the disclosed method apply mutatis mutandis to the system.

The present disclosure furthermore relates to a non-transitory computer readable medium having a computer program with instructions that, on execution on data processing devices of a data processing environment, are set up to carry out the acts of the above-stated method.

All the embodiments described herein may be combined with one another, unless explicitly stated otherwise. Further features, advantages, and possible applications of the present disclosure are revealed by the following description, the exemplary embodiment, and the figures.

### BRIEF DESCRIPTION OF THE DRAWINGS

FIG. 1 depicts an example of a magnetic resonance device.

FIG. 2 depicts a schematic representation of an example of a system for providing position information of an object in a patient tunnel of a magnetic resonance device.

FIG. 3 depicts a schematic representation of an example of a method for providing position information of an object in a patient tunnel of a magnetic resonance device.

FIG. 4 depicts a schematic representation of an example of a patient on a patient table.

FIG. 5 depicts a schematic representation of an example of a patient on a patient table.

FIG. 6 depicts various exemplary sequence parameters that may be used for carrying out a method.

FIG. 7 depicts an example of an acquired magnetic resonance signal.

### DETAILED DESCRIPTION

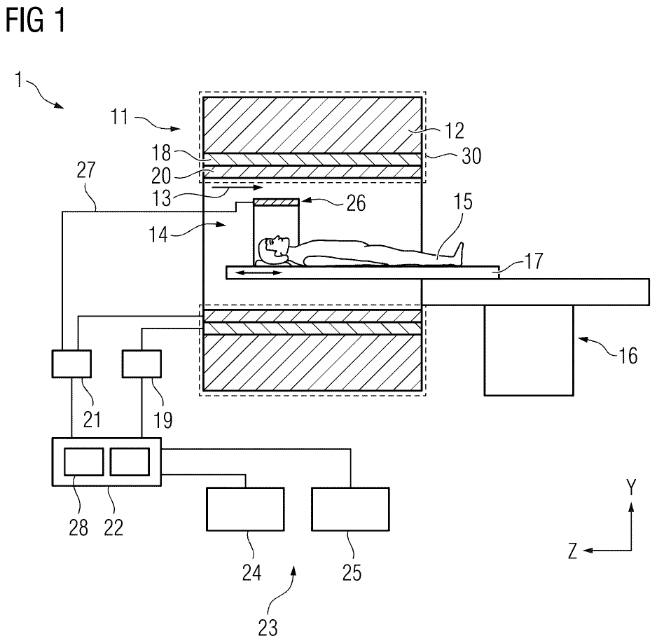

FIG. 1 depicts a magnetic resonance device 1. The magnetic resonance device 1 includes a field generation unit 11 that has a main magnet 12 (B0 magnet) with one or more permanent magnets, electromagnets, or superconductive magnets for generating a strong and in particular uniform main magnetic field 13 (B0 magnetic field). The magnetic resonance device 1 additionally includes a patient tunnel 14 for accommodating a patient 15. In the embodiment shown, the patient tunnel 14 is of cylindrical construction and enclosed in a circumferential direction by the main magnet 12. Configurations of the patient tunnel 14 that deviate from this example are, however, conceivable in principle. The patient tunnel 14 may coincide with an image capture region of the magnetic resonance device 1.

In the example shown in FIG. 1, the patient 15 is positionable in the patient tunnel 14 using a patient positioning apparatus 16 of the magnetic resonance device 1. The patient positioning apparatus 16 has a horizontally movable patient table 17 for this purpose.

The field generation unit 11 has a gradient system with at least one gradient coil 18 for generating a magnetic gradient field that is used for spatial encoding during a magnetic resonance examination. The gradient coil 18 is controlled by way of a gradient control unit 19 of the magnetic resonance device 1. It is conceivable for the gradient system to include a plurality of gradient coils 18 for generating magnetic gradient fields along different spatial directions that may be oriented orthogonally to one another.

The field generation unit 11 moreover includes a radio-frequency system with a radio-frequency coil that in the present exemplary embodiment is configured as a body coil 20 permanently integrated in the magnetic resonance device 1. The body coil 20 is designed to excite nuclear spins that are located in the main magnetic field 12 generated by the main magnet 13. The body coil 20 is controlled by a radio-frequency control unit 21 of the magnetic resonance device 1 and emits radio-frequency excitation pulses into the image capture region, which is formed by the patient accommodation zone 14 of the magnetic resonance device 1. The body coil 20 may furthermore be configured to receive magnetic resonance signals and form a receive unit or part of a receive unit of the magnetic resonance device 1.

The magnetic resonance device 1 has a control unit 22 for controlling the magnetic resonance device 1, in particular the gradient control unit 19 and the radio-frequency control unit 21. The control unit 22 may be configured to coordinate the performance of an imaging sequence, such as a GRE (gradient echo) sequence, a TSE (turbo spin echo) sequence, or a UTE (ultra-short echo time) sequence. The control unit 22 additionally includes a computing unit 28 for evaluating magnetic resonance signals that are acquired with an imaging sequence during a magnetic resonance examination.

The magnetic resonance device 1 may include a user interface 23 that has a signal connection to the control unit 22. Control information, such as imaging parameters of the magnetic resonance examination, may be displayed on a display unit 24, (e.g., at least one monitor), of the user interface 23. The display unit 24 may be configured to provide a graphical user interface with the depiction of a relevant body region of the patient 15. The user interface 23 furthermore has an input unit 25 by way of which magnetic resonance measurement parameters may be input or modified by a user.

The magnetic resonance device 1 may have further components such as a local coil 26. The local coil 26 may be positioned in a position appropriate to the application on a diagnostically or therapeutically relevant region of the body of the patient 15. The local coil 26 may have a plurality of antenna elements that are configured to acquire magnetic resonance signals from the relevant region of the body of the patient 15 and transmit them to the computing unit 28 and/or the control unit 22. The local coil may to this end be connected to the radio-frequency control unit 21 and the control unit 22 by way of an electrical connection lead 27 or another signal connection. Similarly to the body coil 20, the local coil 26 may also be configured to excite nuclear spins in the jaw region 31 of the patient 15. The local coil 26 may be controlled by the radio-frequency control unit 21 for this purpose.

The field generation unit 11 and a magnet retaining structure are conventionally enclosed by a housing 30. The housing 30 may be configured to protect components of the magnetic resonance device 1 from external influences and/or to provide a touch guard for a patient 15.

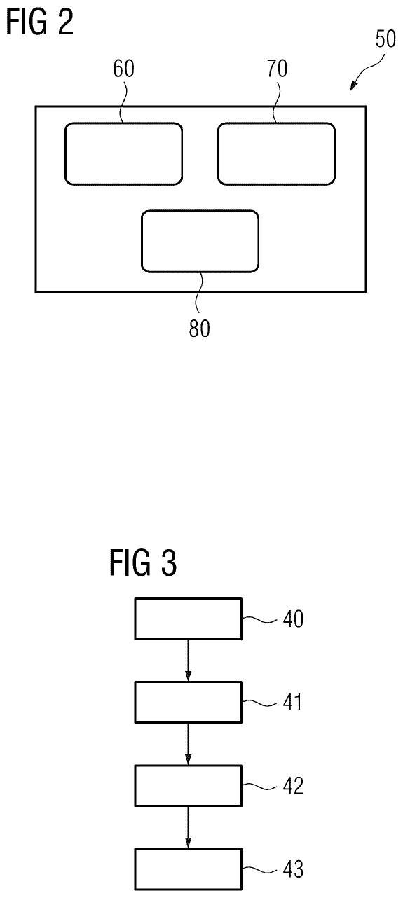

FIG. 2 shows an embodiment of a system 50 for providing position information of an object 31 in a patient tunnel 14 of a magnetic resonance tomograph 100. The system 50 includes a processing circuit 60, a (non-transitory) storage medium 70, and a data interface 80. The (non-transitory) storage medium 70 includes a computer program with instructions that, on execution of the program, cause the processing circuit 60 to carry out a method, wherein the data interface 80 is set up, wherein the data interface 80 is set up to receive the magnetic resonance signal for a predefined slice, e.g., a central transverse slice, which is selected orthogonally to the direction of movement of the object 31 during inward movement of the object 31 into the patient tunnel 14.

FIG. 3 shows a schematic representation of a method for providing position information of an object 31 in a patient tunnel 14 of a magnetic resonance tomograph 100.

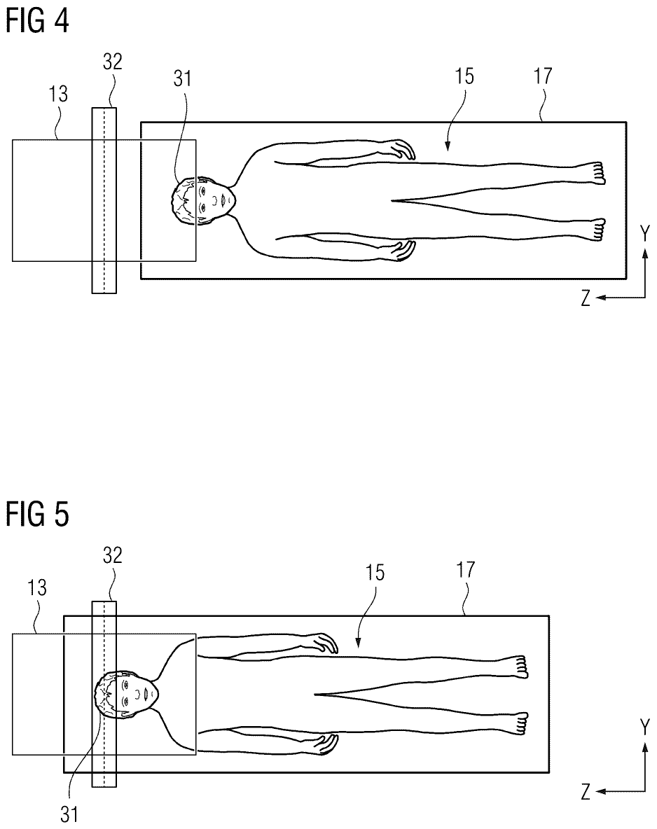

In act 40, a patient 15 is positioned outside the patient tunnel 14, for example, on a patient positioning apparatus 16 with a mobile patient table 17 as shown in FIG. 1. FIG. 4 shows such an arrangement of a patient 15 on a patient table 17. In the embodiment shown, the intention is to determine the position of the head 31 of the patient 15, here, for example, by the initial position of the head 31 along the Z axis in direction of advance. This is one example of a use case of the disclosed method. This is because, if the position of the head 31 of the patient 15 is known, it is possible to provide a comparatively accurate (e.g., cylinder) model of the patient 15 that may in turn be used for monitoring the respective limit values for the absorbed power. The patient table 17 is mounted movably in the Z axis such that the patient 15 may also be moved inward into the patient tunnel 14 in act 41 with the movement of the patient table 17. In the embodiment shown, the patient table 17 and thus the patient 15 is moved inward into the patient tunnel 14 at a speed of 2.0 cm/s.

In act 42, a magnetic resonance signal for a predefined slice 32 is acquired during inward movement of the patient 15 and their head 31 into the patient tunnel 14. As shown in FIGS. 4 and 5, the predefined slice 32 may be a central transverse slice 32 that is selected orthogonally to the direction of movement of the patient 15.

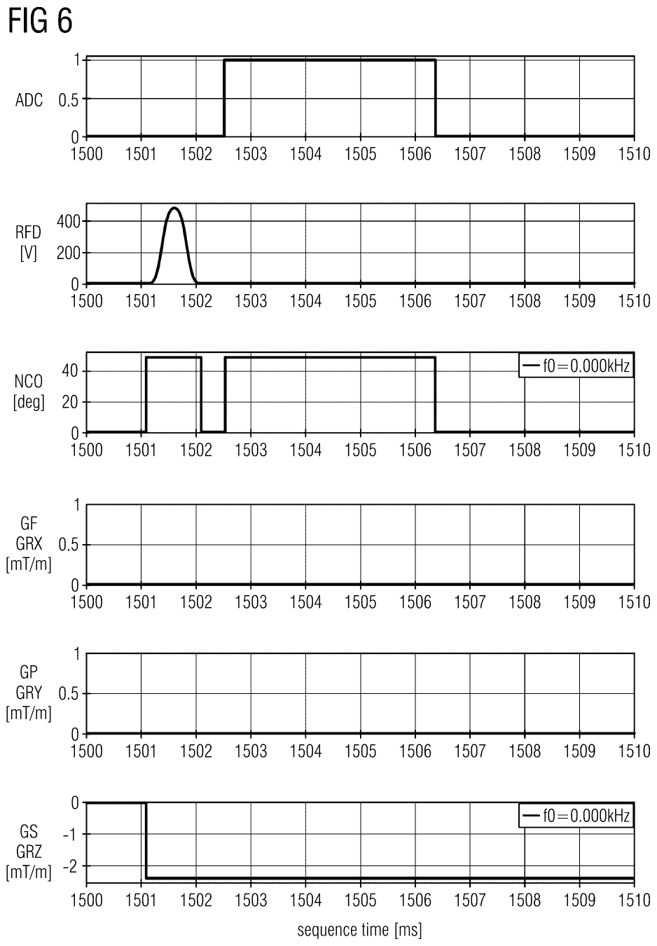

FIG. 6 shows an example of sequence parameters. The present disclosure is not limited thereto.

The Z gradient (cf. GS GRZ) of the magnetic resonance tomograph 100 is set to a constant amplitude of around −2.3 T. The X gradient (cf. GF GRX) and Y gradient (cf. GP GRY) are not switched.

The acquisition window (cf. ADC) amounts to around 4 ms. In one embodiment, the repetition time (TR) amounts to around 1000 ms. Thus, at a speed of advance of the patient 15 into the patient tunnel 14 of 2 cm/s and a repetition time (TR) of 1 s, a position of the head 31 of the patient 15 may be determined with an accuracy of 1 cm. Such an accuracy has proven to be sufficient to enable the provision of a comparatively accurate (cylinder) model of the patient 15. The sequence parameters show here by way of example, as well as the speed at which the patient 15 is moved inward into the patient tunnel 14, may be appropriately configured to enable the provision of other levels of accuracy in position determination.

As shown in FIG. 6, radio-frequency excitation is carried out with just one radio-frequency pulse (cf. RFD). This selected radio-frequency pulse may be comparatively strong such that the radio-frequency power is negligible with regard to patient heating and therefore the specific absorption rates need not be monitored for carrying out the present method.

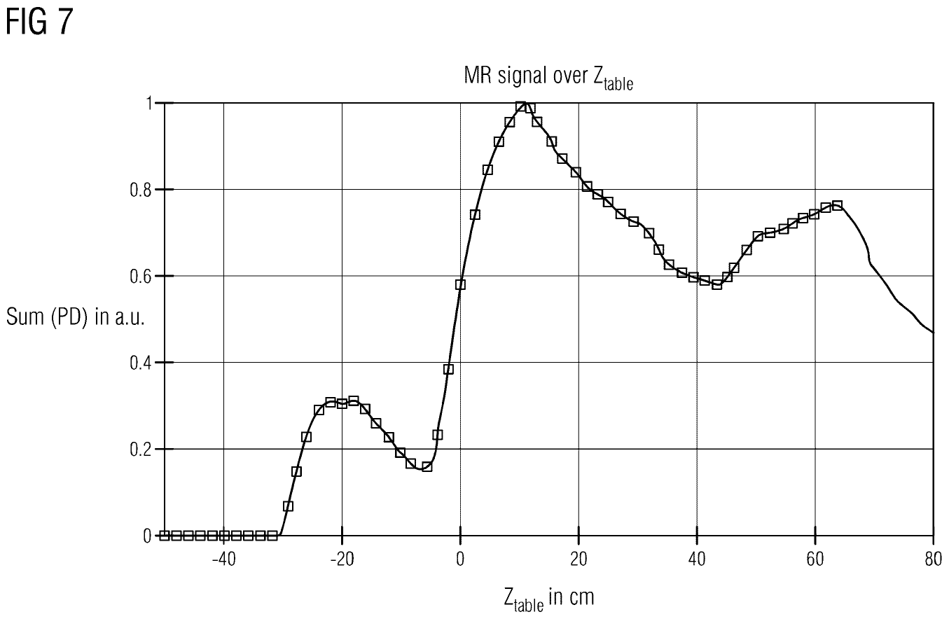

FIG. 7 shows by way of example a magnetic resonance signal that was acquired for the central transverse slice 32 during the inward advance of the patient 15 shown in FIGS. 4 and 5 into the patient tunnel 14. The Y axis shows the magnetic resonance signal normalized to 1 for the central transverse slice 32 and the Y axis shows the Z coordinates in the table coordinate system (TCS) oriented relative to the patient table. As is apparent from the acquired magnetic resonance signal, no magnetic resonance signal is detected for the region from Z=−40 cm to Z=−30 cm and only from Z=−30 cm magnetic resonance may a magnetic resonance signal be detected. Once the patient 15 has been moved inward in head-first position, i.e., with their head 31 in front, into the patient tunnel 14, the rise in the magnetic resonance signal at Z=−30 cm represents the head position of the patient 15. The position of the head 31, here indicated by the beginning of the head 31, may thus be determined in act 43 with the assistance of the acquired magnetic resonance signal. If the intention is merely to determine the head position of the patient 15 in order to enable modeling of the specific absorption rates, the method per se may be terminated at the coordinate Z=−30 cm. Alternatively, or additionally, the complete profile of the patient 15 may also be acquired, for example, to determine the positions of other body parts/regions of the patient 15 so as optionally to enable consistency checks or also to allow volume estimates or the like to be carried out with the assistance of such a patient profile.

The present disclosure is not limited to the embodiment described above providing it is comprised by the subject matter of the following claims. It may additionally be noted that the terms “comprising” and “having” do not exclude any other elements or acts and the indefinite article “a” does not exclude a plurality. It may further be noted that features or acts which have been described with reference to the above exemplary embodiments may also be used in combination with other features or acts.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for providing position information of an object in a patient tunnel of a magnetic resonance tomograph, the method comprising:
arranging the object outside the patient tunnel;
moving the object inward into the patient tunnel along an axis of symmetry of a B0 field magnet of the magnetic resonance tomograph;
acquiring a magnetic resonance signal for a predefined slice during the inward movement of the object into the patient tunnel; and
determining the position information of the object based on the acquired magnetic resonance signal.

2. The method of claim 1, wherein no switching of gradients in the predefined slice is performed during a sequence for the acquiring of the magnetic resonance signal for the predefined slice.

3. The method of claim 1, wherein a slice selection gradient of the magnetic resonance tomograph is applied at a constant amplitude for a predefined duration during a sequence for acquiring the magnetic resonance signal for the predefined slice,
wherein the constant amplitude is in a range of 1 mT/m to 4 mT/m, and
wherein the predefined duration amounts to at least one repetition time of the sequence and is an entire duration of the sequence.

4. The method of claim 1, wherein the object is moved inward into the patient tunnel at a speed of in a range of 0.5 cm/s to 5 cm/s.

5. The method of claim 1, wherein the predefined slice is a transverse slice selected at least substantially orthogonally to a direction of movement of the object, and
wherein at least substantially orthogonally refers to an orientation of the slice that deviates from the transverse or orthogonal by up to ±10° or up to ±5°.

6. The method of claim 1, wherein the predefined slice has a slice thickness in a range of 2 mm to 20 mm.

7. The method of claim 1, wherein a radio-frequency excitation is performed with just one radio-frequency pulse per repetition time.

8. The method of claim 1, wherein an acquisition window is located in a range of 2 ms to 15 ms.

9. The method of claim 1, wherein a repetition time is located in a range of 500 ms to 2000 ms.

10. The method of claim 1, wherein the position information indicates a beginning of the object or an end of the object along the axis of symmetry of the B0 field magnet of the magnetic resonance tomograph.

11. The method of claim 1, wherein the object is a head or a foot of a patient, and
wherein the position information indicates a head position or a foot position of the patient along the axis of symmetry of the B0 field magnet of the magnetic resonance tomograph.

12. The method of claim 11, wherein the position information indicates a beginning of the head or an end of the head of the patient.

13. The method of claim 1, wherein the position information of the object is determined in relation to a patient table.

14. A system for providing position information of an object in a patient tunnel of a magnetic resonance tomograph, the system comprising:
a processing circuit;
a non-transitory storage medium; and
a data interface,
wherein the non-transitory storage medium comprises a computer program with instructions that, on execution of the program, cause the processing circuit to:
move the object inward into the patient tunnel along an axis of symmetry of a B0 field magnet of the magnetic resonance tomograph;
acquire a magnetic resonance signal for a predefined slice during the inward movement of the object into the patient tunnel; and
determine the position information of the object based on the acquired magnetic resonance signal,
wherein the data interface is configured to receive the magnetic resonance signal for the predefined slice during the inward movement of the object into the patient tunnel.

15. A non-transitory computer-readable medium comprising a computer program having instructions that, on execution on a computer device, are configured to:
move an object, which is arranged outside of a patient tunnel, inward into the patient tunnel along an axis of symmetry of a B0 field magnet of a magnetic resonance tomograph;
acquire a magnetic resonance signal for a predefined slice during the inward movement of the object into the patient tunnel; and
determine position information of the object based on the acquired magnetic resonance signal.
